(19) United States
(12) Patent Application Publication
(10) Pub. No.: US 2012/0215311 A1
(43) Pub. Date: Aug. 23, 2012

(54) ARTHROPLASTY SHIM

(76) Inventor: Todd Parry, Saint George, UT (US)

(21) Appl. No.: 13/032,386

(22) Filed: Feb. 22, 2011

(51) Int. Cl. A61F 2/28; A61B 17/56

(52) U.S. Cl. 623/16.11; 606/86 R

(57) ABSTRACT

An arthroplasty shim configured to be inserted into a gap between a bone and a prosthetic joint component. The shim strengthens the prosthetic joint component. The shim can reduce the side-effects of an erroneous bone cut, loss of bone mass or bone failure which may result during revision surgery. The shim can help to balance flexion/extension gaps between bones in the joint and can help balance the joint's varus/valgus alignment. Generally, the shim has a thickness between about 0.5 mm and about 6 mm. The shim may include a spike or lip to anchor or the surface may be cavitated. The shim can be used as a trial to allow for proper fitting of the shim's shape, size and position between the bone and the prosthesis. More than one shim may be used to fill the same gap. Other embodiments are described.

### FIELD

The present application relates to arthroplasty, such as knee replacement arthroplasty.

### BACKGROUND

The proper functioning of a joint, such as the knee, hip, or shoulder, can be impeded by a variety of factors, including, disease, such as osteoarthritis; mechanical injury; bone deformation; and a variety of other factors. Arthroplasty, or the surgical restoration of a joint, is a known procedure that is often used to relieve pain and improve joint function by replacing the diseased or damaged articulating surfaces of a joint with prosthetic components.

One of the most common arthroplasty procedures is knee replacement surgery. Some common forms of knee replacement surgery include total knee replacement (“TKR”) surgery; partial knee replacement surgery, which is also known as unicompartmental arthroplasty (“UKA”); and revision knee surgery.

### SUMMARY

The present application relates to arthroplasty and systems and methods for providing shims in a manner that balances the flexion/extension gaps between bones in a joint and/or that balances the joint's varus/valgus alignment. In some instances, the shims are sized and shaped to be inserted between a bone and a prosthetic joint component. In some instances, the shim is capable of being inserted in a plurality of locations between bone and a corresponding prosthetic component during primary (first time) joint replacement. Moreover, in some instances, the shim is also capable of being inserted between bone and its corresponding prosthetic component in a revision arthroplasty situation, wherein the bone shape after removal of primary components is anatomically different from the native joint. Additionally, in some instances, the described shim is configured to be inserted between a bone and its corresponding prosthetic component without requiring any additional cutting to be done to the bone. In still other instances, the shim is sized and shaped to fit between a variety of anatomically different bone types (e.g., a femur and a tibia) and their corresponding prosthetic components.

In some implementations, the shim includes a tapered portion that extends from a thick peripheral end of the shim thinning towards the shim's central end. In such implementations, the tapered portion may extend along any suitable amount of the shim's length. Additionally, while the tapered portion typically comprises a first face for contacting the bone and a second face for contacting the prosthetic joint component, the first face and the second face can taper towards each other at any suitable taper angle that allows the shim to function as intended. Accordingly, the described shims can accommodate sloped bone cuts (resulting from incorrect bone cuts, bone deformity, or defect from previous component removal) and present a corrected flat surface on which the prostheses can rest. Moreover, the shim can be any suitable thickness. In one non-limiting example, the shim has a maximum thickness between its first and second face that is between about 0.5 mm and about 6 mm.

In some implementations, to prevent stress risers from forming in cement that surrounds the shim, one or more edges (which may include corners) between adjoining surfaces of the shim are rounded. In one non-limiting example, a plurality of edges extending between the shim's peripheral end and the shim's central end are rounded.

In some implementations, the shim comprises an anchor feature that is capable of retaining a portion of the shim over a hard cortical edge of the bone to ensure that the shim is not forced into the spongy medullary tissue of the bone as pressure is applied to the joint. In such implementations, the anchor feature can comprise any suitable component, including, but not limited to, one or more spikes that extend from the shim's first face and a lip that extends from the shim's peripheral end to abut an external perimeter of the bone and/or its corresponding prosthetic component between which the shim is inserted.

While one or more surfaces of the shim can be smooth, in some implementations, the shim comprises a binding feature that is capable of increasing the strength of the bond between cement and the shim. Thus, where the shim is cemented between a bone and a corresponding prosthetic joint component, the binding feature can ensure that the cement holds the shim in place and prevents it from becoming dislodged as the joint functions. In one non-limiting example, the binding feature comprises one or more holes or voids that are placed in the shim to permit cement to pass through and bind to the shim. In another non-limiting embodiment, one or more surfaces of the shim comprises grooves or a texture that help maintain cement contact between the surface of the shim and a surface of a bone and/or prosthetic component.

While the methods and processes described herein may be particularly useful in the area of knee arthroplasty, those skilled in the art can appreciate that they can be used in a variety of different applications and in a variety of different areas of arthroplasty. Some non-limiting examples of such applications and areas include, hip arthroplasty, shoulder arthroplasty, ankle arthroplasty, vertebral arthroplasty, and any other suitable joint replacement or repair procedure.

These and other features and advantages will be set forth or will become more fully apparent in the description that follows and in the appended claims. These features and advantages may be realized and obtained by means of the instruments and combinations particularly pointed out in the appended claims. Furthermore, these features and advantages may be learned by practicing the systems and methods of providing the shims.

### BRIEF DESCRIPTION OF THE DRAWINGS

In order that the manner in which the above recited and other features and advantages are obtained, a more particular description will be rendered by reference to specific embodiments thereof, which are illustrated in the appended drawings. Understanding that the drawings depict only typical embodiments and are not, therefore, to be considered as limiting the scope of the claims, the embodiments will be described and explained with additional specificity and detail through the use of the accompanying drawings in which:

FIG. 1 illustrates a side-plan view of a knee joint comprising prior art prosthetic components;

FIGS. 2A-2B each illustrates a side-plan view of a representative embodiment of an arthroplasty shim;

FIGS. 3A-3C each illustrates a side-plan view of a representative embodiment of the arthroplasty shim;

FIGS. 4A-4C each illustrates a face-plan view of a representative embodiment of the arthroplasty shim;

FIGS. 5A-5B each illustrates a face-plan view of a representative embodiment of the arthroplasty shim, wherein a perimeter of the shim defines a recess;

FIG. 6 illustrates a perspective view of a representative embodiment of the shim, wherein each of the edges between surfaces of the shim is rounded;

FIG. 7A illustrates a side-plan view of a femur showing non-limiting examples of suitable locations in which a representative embodiment of the described arthroplasty shim can be inserted between the femur and a prosthetic component;

FIG. 7B illustrates a face view of a tibia, showing non-limiting examples of suitable locations in which a representative embodiment of the described arthroplasty shim can be inserted between the tibia and a prosthetic component;

FIG. 7C illustrates a side view of some representative embodiments of the arthroplasty shim;

FIG. 7D illustrates a face view of a representative embodiment of the arthroplasty shim;

FIG. 8A illustrates a face view of a proximal-tibial surface having a representative embodiment of the shim disposed thereon;

FIG. 8B illustrates a side-plan view of a representative embodiment of the arthroplasty shim comprising a spike;

FIG. 8C illustrates a side-plan view of a representative embodiment of the arthroplasty shim comprising a lip;

FIG. 9 illustrates a flow chart depicting a representative embodiment of a method for using the arthroplasty shim; and

FIG. 10 illustrates cross-sectional view of a representative embodiment of an impact-driven cement applicator.

FIG. 11A illustrates side plan views of an alternative exemplary embodiment of an arthroplasty means.

FIG. 11B illustrates side plan views of an alternative exemplary embodiment of an arthroplasty means.

FIG. 11C illustrates side plan views of an alternative exemplary embodiment of an arthroplasty means.

FIG. 11D illustrates side plan views of an alternative exemplary embodiment of an arthroplasty means.

FIG. 11E illustrates side plan views of an alternative exemplary embodiment of an arthroplasty means.

FIG. 11F illustrates side plan views of an alternative exemplary embodiment of an arthroplasty means.

FIG. 12A illustrates side plan views of an alternative exemplary embodiment of an arthroplasty means.

FIG. 12B illustrates side plan views of an alternative exemplary embodiment of an arthroplasty means.

FIG. 12C illustrates side plan views of an alternative exemplary embodiment of an arthroplasty means.

FIG. 12D illustrates side plan views of an alternative exemplary embodiment of an arthroplasty means.

FIG. 12E illustrates side plan views of an alternative exemplary embodiment of an arthroplasty means.

FIG. 12F illustrates side plan views of an alternative exemplary embodiment of an arthroplasty means.

FIG. 12G illustrates side plan views of an alternative exemplary embodiment of an arthroplasty means.

FIG. 12H illustrates side plan views of an alternative exemplary embodiment of an arthroplasty means.

FIG. 12I illustrates side plan views of an alternative exemplary embodiment of an arthroplasty means.

FIG. 12J illustrates side plan views of an alternative exemplary embodiment of an arthroplasty means.

FIG. 12K illustrates side plan views of an alternative exemplary embodiment of an arthroplasty means.

FIG. 12L illustrates side plan views of an alternative exemplary embodiment of an arthroplasty means.

FIG. 12M illustrates side plan views of an alternative exemplary embodiment of an arthroplasty means.

FIG. 12N illustrates side plan views of an alternative exemplary embodiment of an arthroplasty means.

FIG. 12O illustrates side plan views of an alternative exemplary embodiment of an arthroplasty means.

FIG. 12P illustrates side plan views of an alternative exemplary embodiment of an arthroplasty means.

FIG. 12Q illustrates side plan views of an alternative exemplary embodiment of an arthroplasty means.

FIG. 12R illustrates side plan views of an alternative exemplary embodiment of an arthroplasty means.

FIG. 12S illustrates side plan views of an alternative exemplary embodiment of an arthroplasty means.

FIG. 12T illustrates side plan views of an alternative exemplary embodiment of an arthroplasty means.

### DETAILED DESCRIPTION

The present application relates to arthroplasty. In particular, the present application relates to systems and methods for providing shims that are sized and shaped to be inserted between a bone and a prosthetic joint component. The shims can be sized and shaped so they can be inserted at any suitable location between a bone surface and a prosthetic joint component that allows the shim to balance flexion/extension gaps between bones in the joint and/or to balance the joint's varus/valgus alignment. In some instances, one or more shims retain a desired distance between the cut bone and the prosthetic component while cement around the shim hardens. Additionally, unlike other methods that require additional bone to be removed in order to balance the flexion/extension gaps in a joint and/or to balance the joint's varus/valgus alignment, in some embodiments the described shims are used to balance the joint without requiring any additional bone to be removed to accommodate one or more of the shims.

The described shim can be used in virtually any arthroplasty procedure in which it is beneficial to balance flexion/extension gaps between bones in the joint and/or to balance the joint's varus/valgus alignment. Some non-limiting examples of suitable arthroplasty procedures include surgeries for a total or partial replacement of a knee (including a UKA of a medial or lateral compartment), hip, shoulder, vertebrae, elbow, ankle, wrist, or any other suitable synovial joint found in the body. For simplicity, however, the following discussion focuses on using the shim in complete or partial knee replacements.

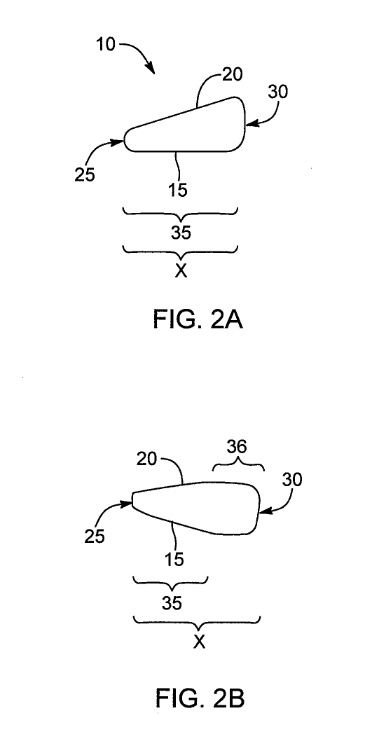

The shim can comprise any suitable component or characteristic that allows it to be inserted between a low end of a bone and a prosthetic joint component so as to balance the flexion/extension gaps in the joint (e.g., the knee) and/or to balance the joint's varus/valgus alignment. By way of non-limiting illustration, FIG. 2A illustrates an embodiment in which the shim 10 comprises a first face 15 for contacting a bone surface, a second face 20 for contacting a surface of a prosthetic joint component, a central end 25, and a peripheral end 30.

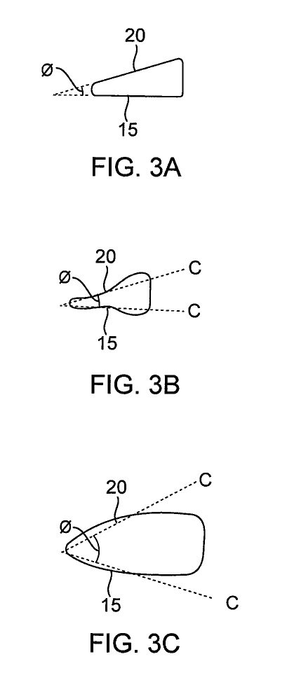

FIGS. 2A and 2B show that, according to some embodiments, the first face 15 and the second face 20 are sloped towards each other to form a wedge-shaped tapered portion 35 that extends from the shim's central end 25, towards the shim's peripheral end 30. In such embodiments, the tapered portion can perform any suitable function, including without limitation, allowing the shim to be easily inserted between a bone and a corresponding prosthetic component that is seated on the bone. As used herein, the term seated may refer to a prosthetic component that is placed over a cut surface of a bone at a joint in the intended manner. It should be noted that the term seated does not necessarily connote that the prosthetic component is cemented or otherwise fixedly connected to the bone on which it is placed.

Where the shim 10 comprises the tapered portion, the tapered portion can extend for any suitable length of the shim. Indeed, in some embodiments, FIGS. 2A and 2B illustrate that the tapered portion 35 extends more than: about 5%, about 10%, about 15%, or about 20% of the distance X between the shim's central end 25 and peripheral end 30. Along these lines, in some embodiments, the tapered portion extends less than: about 100%, about 80%, about 60%, or about 50% of the distance X between the shim's central end and peripheral end. For instance, while FIG. 2A illustrates a non-limiting embodiment in which the tapered portion 35 extends approximately the entire distance X between the shim's central 25 and peripheral 30 ends, FIG. 2B illustrates another non-limiting embodiment in which the tapered portion 35 extends less than about 70% of the distance X between the shim's central 25 and peripheral 30 ends. Accordingly, FIG. 2B shows the shim 10 can comprise a plateau portion 36.

Where the shim's first face 15 and second face 20 form a tapered portion 35, the first face and second face can be shaped to have any suitable profile that allows the shim 10 to be inserted between a bone and a prosthetic joint component that is seated at the joint end of the bone. Some examples of suitable profile shapes include, but are not limited to, a flat, a convex, a concave, and an irregular shaped profile. By way of illustration FIGS. 3A, 3B, and 3C respectively illustrate non-limiting embodiments in which the first 15 and second 20 faces of the tapered portion 35 of the shim 10 comprise a flat profile, a concave profile, and a convex profile.

Where the first face 15 and the second face 20 form a tapered portion 35, the taper angle between the first face 15 and the second face may be any suitable angle that allows the shim 10 to be wedged in between a bone and a prosthetic joint component seated at a cut end of the bone. In some embodiments, FIGS. 3A through 3C show the taper angle θ between the first face 15 and the second face 20 (including the taper angle between the slope of the curvature C of the first face 15 and the second face 20) is less than: about 50 degrees, about 45 degrees, about 35 degrees, or about 25 degrees. Additionally, in some embodiments, FIGS. 3A through 3C show the taper angle θ between the first face 15 and the second face 20 (including the angle between the slope of curvature C of the first face 15 and second face 20) is greater than: about 1 degree, about 10 degrees, about 15 degrees, and about 20 degrees.

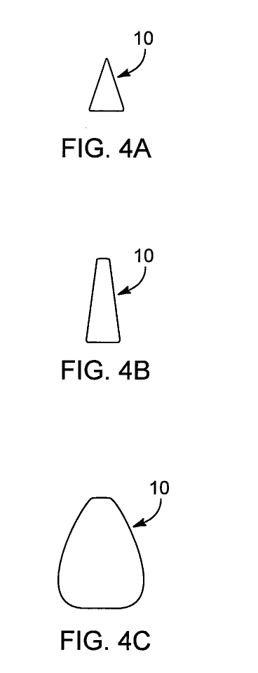

From a view of the shim's first 15 and/or second face 20, the shim 10 can have any suitable shape that allows it to be inserted in a plurality of locations between a bone and a corresponding prosthetic joint component that is seated at the joint end of the bone. By way of non-limiting example, from its face view, the shim can comprise a shape that is substantially square, rectangular, triangular, trapezoidal, polygonal, rounded, irregular, horseshoe-shaped, comma-shaped, or any other suitable shape. For instance, FIGS. 4A through 4C respectively illustrate embodiments in which the shim 10, from its face view, comprises a triangular shape, a trapezoidal shape, and a rounded shape.

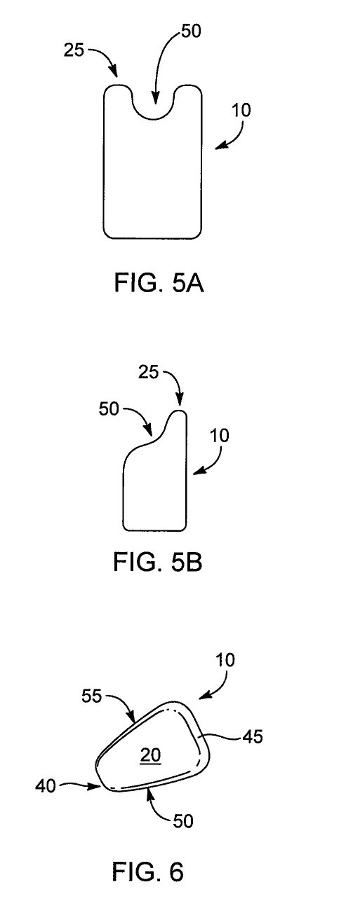

In some embodiments, the shim 10 defines a recess in a portion of its perimeter that is disposed at or towards the shim's central end 25. In such embodiments, the recess can serve any suitable purpose, including, but not limited to, allowing the shim to fit around at least a portion of any suitable object (including without limitation, a pin, stem, screw, keel, protrusion, etc.) that extends from a prosthetic joint component into a bone, without having the object pass through an aperture in the shim. Accordingly, in some embodiments, the shim is capable of being inserted into and/or being retracted from a space between a bone and its corresponding prosthetic joint component so that a portion of the shim extends past at least a portion of the object, while the object is already extended into the bone.

Where a perimeter of the shim 10 defines recess, the recess can give the shim any suitable appearance that allows the shim to function as intended. By way of non-limiting example, FIG. 5A illustrates an embodiment in which the recess 50 gives a U-shaped appearance to a portion of the shim 10. In another non-limiting example, FIG. 5B illustrates an embodiment in which the recess 50 provides a portion of the shim 10 with a Utah-shaped appearance, or an uneven appearance in which a corner of the shim 10 is missing.

In some embodiments, one or more of the edges (including corners) between surfaces on the shim 10 are optionally rounded so as to not have a straight edge (or vertex) between them. In such embodiments, the rounded edges may serve any suitable purpose, including, but not limited to, reducing or preventing stress risers from forming in the cement that surrounds the shim.

Where the shim 10 comprises one or more rounded edges, any suitable edges of the shim can be rounded. By way of non-limiting example, where the shim 10 comprises the first face 15, the second face 20, a central face 40, a peripheral face 45, a first side 50, and a second side 55 (as illustrated by FIG. 6), the edge between the first face 15 and the first side 50; the edge between the first face and the second side 55; the edge between the second face 20 and the first side 50; the edge between the second face 20 and the second side 55; one or more edges between the peripheral face 45 and the first face 15, the second face 15, the first side 50, and/or the second side 55; and/or one or more edges between the central face 40 and the first face 15, the second face 20, the first side 50, and/or second side 55 are rounded. Specifically, FIG. 6 shows a non-limiting embodiment in which each of the edges between the shim's surfaces (e.g., the first face 15, second face 20, central face 40, peripheral face 45, first side 50, and second side 55) is rounded.

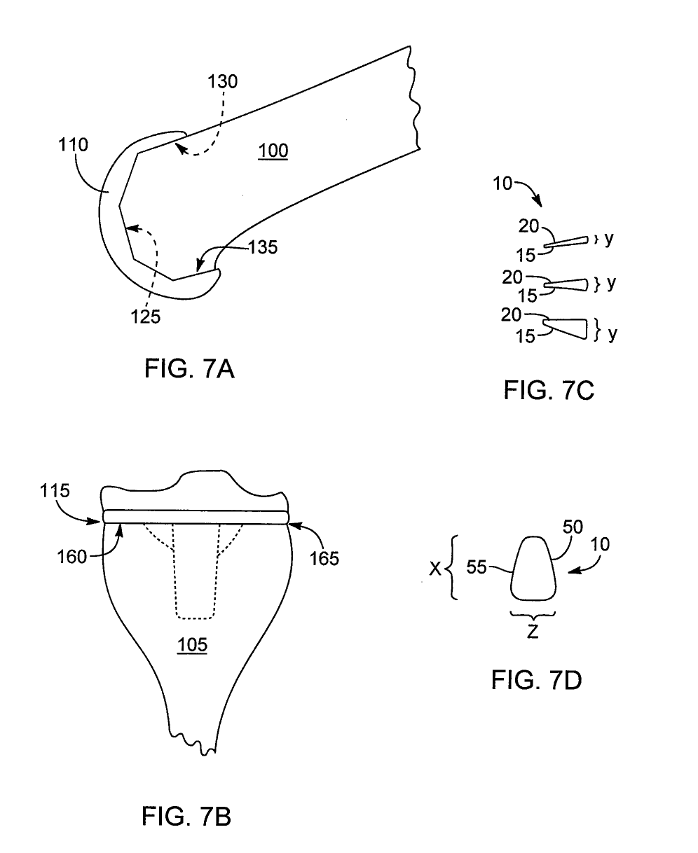

The shim can be any suitable size that allows it to both be inserted between a variety of anatomically different bone types and their corresponding prosthetic joint components, as well as to be inserted at a plurality of locations between a bone and a prosthetic joint component seated on the bone. The size of the shim may be determined by at least one of the following factors: the physical dimensions of the shim, the shape of the shim or the position of the shim in relation to the void or gap being filled between the prosthetic joint component and the bone. In some embodiments, the described shim is sized and shaped to fit between a variety of anatomically different types of joint bones and their corresponding prosthetic joint components. In one non-limiting example, FIG. 7A depicts an embodiment in which the shim 10 is configured to be placed between the femur 100 and the femoral component 110 as well as to be placed between the tibia 105 and the tibial component 115, whether or not the prosthetic components comprise primary or revision components, for either a TKR or a UKA.

Similarly, in some embodiments, the described shim 10 is able to be placed between a bone and a prosthetic component seated thereon at a plurality of locations. In such embodiments, the shim is sized and shaped to be inserted at any suitable location between the bone and the prosthetic component that allows the shim to help balance the joint's flexion/extension gaps and/or varus/valgus alignment. In one non-limiting example, FIG. 7A shows an embodiment in which the shim 10 is capable of being inserted between the femoral prosthetic component 110 and the femur's distal surface 125, anterior surface 130, and/or posterior surface 135 of the medial and/or lateral side of the knee. In another non-limiting example, FIG. 7B shows an embodiment in which the shim 10 is sized to be placed between the tibia 105 and the tibial prosthetic component 115, at an anterior edge 160 and/or a posterior edge (not shown) that is located on the medial and/or lateral side of the knee. In addition to the locations illustrated in FIGS. 7A and 7B, the shim 10 can be sized and shaped to be placed at any suitable location between the femur and the femoral component and/or the tibia and the tibial component that allows the shim to be inserted in and/or extracted from that location when the prosthetic components are seated in their appropriate locations.

Where the shim is shaped and sized to be inserted at a plurality of locations between a bone and a prosthetic component and/or to be used with a variety of bone types, the shim can help balance the joint's flexion/extension gaps and/or the joint's varus/valgus alignment in any suitable manner. In one non-limiting example, where the flexion gap at a medial edge of the knee is larger than the corresponding extension gap, the flexion/extension gaps are balanced by inserting an appropriately sized shim between the femur's posterior surface 135 and the femoral component 110, at the femur's medial edge or at the posterior medial tibial bone/prosthesis interface. In another non-limiting example, where the knee's varus/valgus alignment is unbalanced so that the leg has a varus or bow-legged alignment, the alignment is balanced by inserting an appropriately sized shim between femur's distal surface 125 and the femoral component 110, from the knee's medial side.

With respect to the specific size of the shim 10, at its maximum thickness, or the largest distance between its first face 15 and second face 20 (shown as Y in FIG. 7C), the shim can be any suitable thickness that allows it to balance a joint's flexion/extension gaps and/or varus/valgus alignment. In some embodiments, at its thickest point Y, the shim 10 is less than: about 6 mm, about 5 mm, about 4 mm, about 3 mm, about 2 mm, or about 1 mm thick. Similarly, in some embodiments, at its thickest point Y, the shim is more than: about 0.5 mm, about 1 mm, about 2 mm, about 3 mm, or about 4 mm thick. Indeed, in some presently preferred embodiments, the shim is available in a variety of sizes, including, but not limited to, sizes having a thickest point Y of 1 mm±0.5 mm, 2 mm±0.5 mm, 3 mm 0.5 mm, and/or 4 mm±0.5 mm.

At its widest point, or the maximum distance between its first 50 and second 55 sides (as shown by Z in FIG. 7D), the shim 10 can be any suitable width that allows it to function as intended. Indeed, in some embodiments, at its widest point Z, the shim is less than: about 2 centimeters (“cm”), about 1.5 cm, about 1 cm, about 8 mm, about 6 mm, or about 4 mm wide. Furthermore, in some embodiments, at its widest point Z, the shim is more than: about 2 mm, about 4 mm, about 6 mm, or about 8 mm wide. In one non-limiting example, the shim's maximum width Z is between about 4 mm and about 1 cm.

At its maximum length, or the maximum distance between its central 25 and peripheral 30 ends (as shown by X in FIG. 7D), the shim can be any suitable length X that allows it to function as intended. In some embodiments, the shim's length X is less than: about 2 cm, about 1.5 cm, or about 1 cm. Along these lines, in some embodiments, the shim's length is more than: about 2 mm, about 4 mm, or about 6 mm. In one non-limiting example, the shim's maximum length X is between about 1 and about 1.5 centimeters.

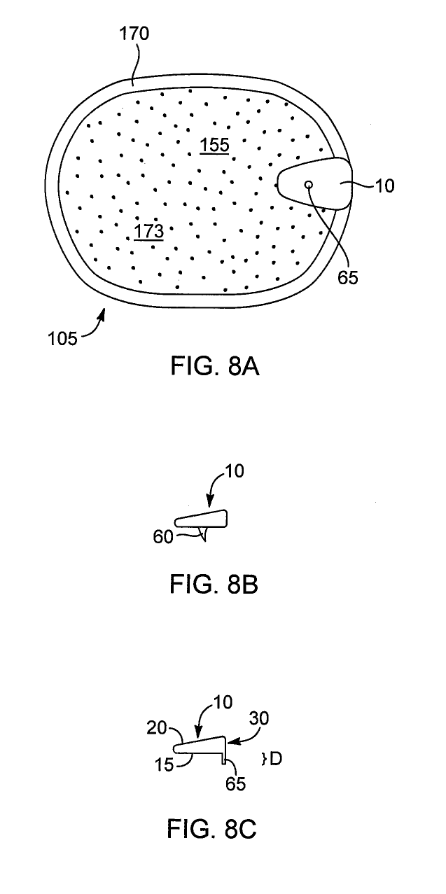

In addition to the aforementioned characteristics, the shim 10 can comprise any other suitable component or characteristic that allows it to function as intended. Indeed, in some embodiments, the shim comprises an anchor feature that is capable of retaining the shim 10 at a hard cortical edge (170 as shown in FIG. 8A) of a bone (e.g., tibia 105) when pressure is applied to the joint. In such embodiments, the anchor feature can comprise any suitable component that is capable of securing the shim at a bone's cortical edge and preventing the shim from being dislodged from its desired position when pressure is exerted on the shim, such as during the impaction of the overlying knee prosthesis or with the pressurization of a cement, such as methyl-methacrylate, at the time of implantation. Accordingly, such an anchor feature can maintain the shim at an outer cortical rim of the bone during the implantation/cementing process so that the shim does not slip off this outer cortical rim of hard bone into the softer, cancellous, inner bone, thus allowing the shim to compact the soft bone and to no longer maintain the original gap that it held during its proper trial positioning. Some non-limiting examples of suitable anchor features include one or more spikes extending shim's first face 15 and a lip extending from the shim's peripheral end 30.

Where the anchor feature comprises at least one spike extending from the shim's first face 15, the spike can have any suitable characteristic that allows it to be forced into the cancellous bone so as to retain a portion of the shim 10 over the hard cortical edge 170 to prevent the shim from moving into the spongy medullary bone 173 and subsiding or from moving out of the joint when the cement is pressurized during component application and impaction. By way of non-limiting example, the spike can be any suitable length. In another non-limiting example, the spike can be disposed any suitable distance from the shim's peripheral end that allows the spike to be embedded within the medullary bone while a suitable amount of the shim rests on the bone's cortical edge. In still another non-limiting example, the spike can have any suitable shape. For instance, while the spike can be straight, FIG. 8B shows a non-limiting embodiment in which the spike 60 is curved to have a barb-like appearance.

Where the anchor feature comprises a lip that extends from the shim's peripheral end 30, the lip may have any characteristic that allows it to function as intended. By way of non-limiting example, the lip can extend from shim's peripheral end 30 on the side of the first face 15 and/or the side of the shim's second face 20. For instance, FIG. 8C illustrates a non-limiting embodiment in which the shim 10 has a lip 65 that extends from the shim's peripheral end 30, on the side of the shim's first face 15. In another non-limiting example, the lip 65 can extend any suitable amount past the shim's first 15 and/or second 20 face. In some embodiments, for instance, the lip extends a distance (shown as D in FIG. 8C) of less than: about 1 cm, about 5 mm, about 3 mm, about 2 mm, or about 1 mm. Additionally, in some embodiments, the lip extends a distance D of more than: about 0.5 mm, 1 mm, or 2 mm.

In some embodiments, the shim 10 comprises a binding feature that allows any suitable cement (such as methyl-methacrylate) or bone to infiltrate into the shim and increase the strength of the bond between the shim and the cement that attaches the prosthetic component to the bone. Some non-liming examples of suitable binding features include holes that extend into and/or through the shim as well as a surface (including, but not limited to, the first face, second face, first side, and/or second side) that is grit-blasted, knurled, ridged, grooved, notched, roughened, or that is otherwise textured to allow a suitable cement to partially infiltrated into pores, abrasions, or other features of the shim. By way of illustration, FIG. 8A shows a non-limiting embodiment in which the binding feature comprises a hole 65.

While some embodiments of the shim 10 are intended to be implanted into a joint, other embodiments of the shim are intended to be used as trial shims. In such embodiments, the trial shim may serve any suitable purpose. In one non-limiting example, the use of trial shims allows a surgeon (which is used herein to include any suitable user, such as a nurse, an assistance, a technician, etc.) to use shims of a variety of sizes to balance the joint's flexion/extension gaps and/or varus/valgus alignment before the prosthetic components are fixedly connected to the appropriate bones. In this example, once the user has found one or more trial shims of a proper size, shape, and thickness and has placed those shims in proper locations to balance the joint, the surgeon can replace the trial shims with one or more corresponding implantable shims, such as a shim with a spike 60.

Where the shim comprises a trial shim, the trial shim may comprise any component or characteristic that allows it to be used to properly balance a joint while allowing the shim to be inserted into and/or extracted from its desired position when the appropriate prosthetic component is seated on the bone. In one non-limiting example, where the shim comprises a trial shim, the first face of the shim is free from spikes or other protrusions that may prevent the trial shim from being pulled from between the bone (e.g., the femur) and its corresponding prosthetic component (e.g., the femoral component) when the prosthetic component is seated on the bone.

The shim 10 can be made of any suitable material that allows it to function as intended. Some examples of suitable materials include, but are not limited to, metals such as steel, titanium, cobalt chrome, stainless steel, tebecular metals, or any other suitable metal, as well as alloys of these metals; a plastic, such as polyethylene, polymethylmethacrylate, bone cement with or without an antibiotic additive, plastics compatible with bone cement, or any other suitable plastic; a suitable ceramic material; a suitable composite material such as carbon-based composites; or any other material allows the shim to function as intended. Indeed, in some embodiments in which the shim is implantable, the shim comprises titanium. In some embodiments where the shim is a trial shim, the shim comprises an autoclavable material and/or a low cost material that is intended to be discarded after a single use (e.g., a plastic). Accordingly, in such embodiments, the trial shim can be sterilized, reused, and/or discarded, as desired.

Materials properties which promote bone ingrowth, based both on texture, cavitation, voids and surface chemistry are also considered as the implant is evaluate. The surface of the shim material may be chemically treated with anti-biotic, adhesive, cement binder, or to give the surface a desired property including making it hydrophobic or hydrophilic.

The shim 10 can also be made in any suitable manner. Indeed, the shim can be made through a process that includes molding, extruding, cutting, grinding, etching, pressing punching, casting, molding or otherwise forming the shim.

In some embodiments, the shim is manufactured in a kit that comprises a variety of different shim sizes and/or shapes. Accordingly, in such embodiments, the surgeon can try to balance the joint with a variety of trial shims of different sizes and shapes. After finding the appropriate trial shim, the surgeon can insert an implantable shim of the same size and shape as the trial shim. In this manner, the surgeon can find an appropriate shim without dirtying multiple implantable shims in the process.

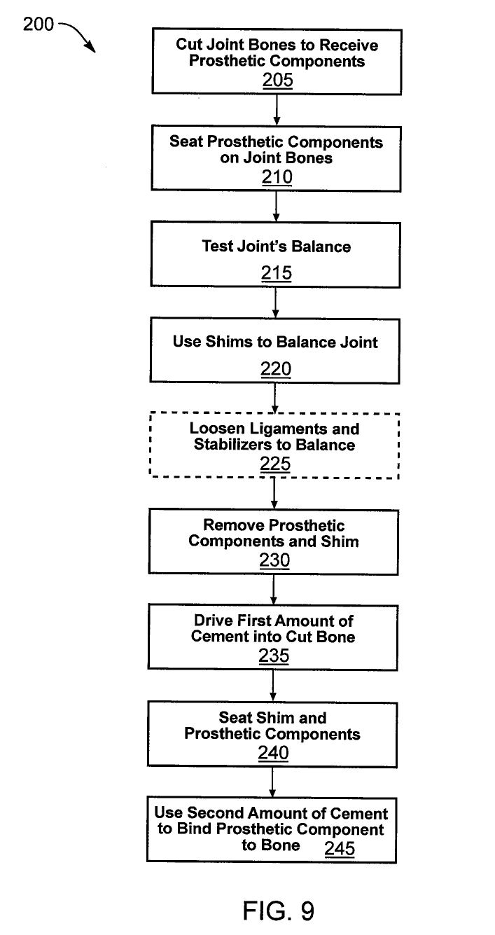

The shim 10 can be used in any suitable manner that allows it to balance a joint's flexion/extension gaps and/or varus/valgus alignment. By way of non-limiting example, FIG. 9 illustrates a flow chart depicting a representative method 200 for using the shim 10. It should be noted, however, that the method 200 shown in FIG. 9 can be modified in any suitable manner. For instance, one or more elements can be added to, be removed from, be repeated, or be rearranged within the method 200 in any suitable manner that allows the shim to balance a joint.

With reference to FIG. 9, that figure shows that the illustrated method 200 begins at step 205 by preparing the bones of the joint to receive the appropriate prosthetic components (such as a femoral and/or a tibial prosthetic component). After the bones have been cut, step 210 shows the method 200 continues as the appropriate prosthetic components (e.g., trial components) are seated on the cut portions of the bones. With the prosthetic components in place, step 215 shows that the surgeon tests the joint to see if the joint's flexion/extension gaps and/or varus/valgus alignment are properly balanced. Where the joint is not properly balanced, step 220 shows the surgeon balances the joint by placing one or more shims (e.g., trial shims) between one or more bones in the joint and the bones' corresponding prosthetic components.

As previously mentioned, the shim can be placed in any suitable location that allows the shim to balance the joint. As a general rule, however, the shim is placed where the bone is lower than desired (e.g., at the bone's low end). Accordingly, the surgeon balances the joint by using the shim to increase the distance between one or more cut surfaces of one or more bones in the joint and its portion of an overlying prosthesis.

Continuing with the method, step 225 shows that the surgeon optionally loosens ligaments and/or static stabilizers around the joint to fine tune the balance of the joints flexion/extension gaps and/or varus/valgus alignment. It should be noted, however, that because the shim is used to build up the bone and increase the distance between a joint bone and its corresponding prosthetic component, the ligaments and/or static stabilizers may be loosened less than if the knee were balanced completely by loosening the ligaments and static stabilizers. It should also be noted, that in some embodiments, in order to finely adjust the joint's balance and alignment, steps 220 and 225 can be repeated one or more times, in any order.

Once the surgeon has determined where to put the shim and the specific size and shape of the shim needed to balance the joint, step 230 shows the surgeon removes the shim and the prosthetic components.

Next at step 235, FIG. 9 shows the method 200 continues as a first amount of cement is driven into the cancellous bone that is to be fixedly connected with the appropriate prosthetic component. Although in many conventional arthroplasty surgeries, cement is often driven into the cancellous bone as the prosthetic components are pounded down onto a bone surface, in some instances, the described shim would cover a portion of the bone and prevent cement from properly penetrating into the bone. As a result, some non-limiting embodiments of the current method include forcing a first amount of cement into the cancellous bone before the implantable shim is placed between the bone and a corresponding prosthetic component.

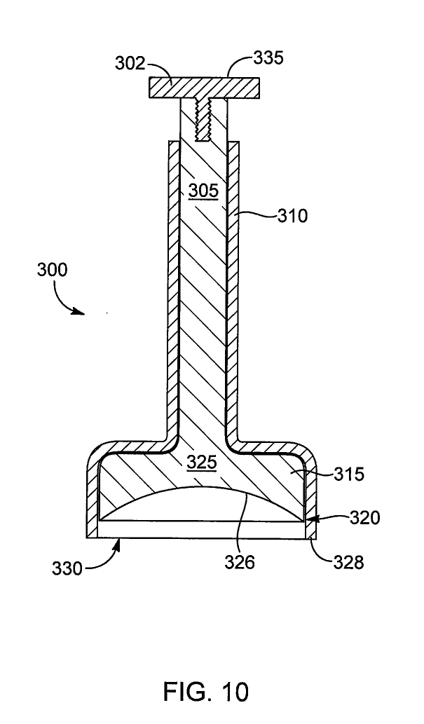

This first amount of cement can be driven into the bone in any suitable manner, including without limitation, through the use of an impact-driven cement applicator. Where an impact-driven applicator is used to apply cement to a bone, the applicator can have any suitable component. By way of non-limiting illustration, FIG. 10 illustrates an embodiment in which the applicator 300 comprises a removable head 302 that is connected to a shaft 305 that runs through a handle 310 to a piston head 315, which is disposed within a chamber 320 containing cement 325. Alternative exemplary embodiments teach filling the concave down cavity with cement and then driving the cement into the bone by striking the back of the head 305. While the piston head's proximal end can be substantially flat, FIG. 10 shows that, in some other non-limiting embodiments, the proximal end 326 of the piston head 315 comprises a concave surface that allows the piston head 315 to hold a relatively large amount of cement before impact.

In addition to the aforementioned components and characteristics, the impact-driven cement applicator can have any other suitable component or characteristic. In one non-limiting example, the piston head 315 can have any suitable shape from its face view (including, without limitation, a circular, square, polygonal, or irregular shape) that allows it to function as intended. Indeed, in some non-limiting embodiments, the piston head (from its face view) is shaped to match and form a seal with the portion of the bone into which it will be used to drive cement.

Where the first amount of cement is driven into cancellous bone with an impact-driven cement applicator, the applicator can be used in any suitable manner. In one non-limiting example, the surgeon places the chamber's open end 330 over a cut surface of the bone (not shown in FIG. 10) to form a seal between the bone and the applicator. After forming the seal, the surgeon holds the applicator's handle and strikes the head 302 at the distal end 335 of the shaft 305 with a hammer or another suitable object. As the surgeon strikes the head, the piston head drives cement from the chamber down into the cancellous bone.

Returning to FIG. 9, step 240 shows the method 200 can continue as the appropriate shim (e.g., an implantable shim) and prosthetic components (e.g., implantable prosthetics) are seated on the bone. As a final step in the illustrated method 200, step 245 shows that a second amount of cement is added between the bone and the prosthetic component to secure the bone and the prosthetic component together.

The systems and methods for providing the shims for arthroplasty described above can provide features that are not offered by the common forms of knee replacement surgery include total knee replacement (“TKR”) surgery, partial knee replacement surgery (also known as unicompartmental arthroplasty or “UKA”), and revision knee surgery. Generally, in a TKR, the femur's lateral and medial condyles, or the articulating surfaces at the femur's distal end, are removed and replaced with a femoral prosthetic component. Additionally, in a TKR, the tibial plateau at the tibia's proximal end is often removed and replaced with a tibial prosthetic component. In contrast, during a UKA, the knee is generally divided into three compartments—namely a medial compartment that is located at the inside of the knee, a lateral compartment that is located at the outside part of the knee, and a patellofemoral compartment that is located between the kneecap and the femur. In a UKA where the damage is confined primarily to one compartment (namely the medial or lateral compartment), the articulating surfaces from that particular compartment of the femur and/or tibia are usually removed and replaced with prosthetic components. With respect to revision knee surgery, such surgeries generally involve removing one or more prosthetic components that were previously placed within the knee (“primary components”) but have become worn, did not fit properly, or have otherwise prevented the knee from functioning properly, and replacing the primary components with one or more replacement components (“revision components”).

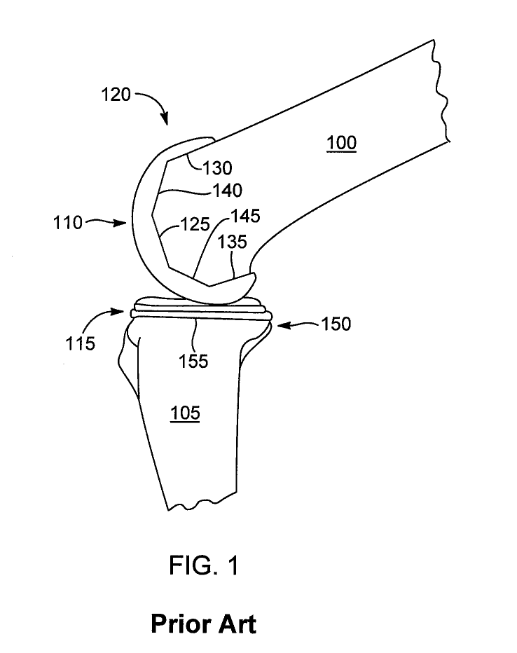

While there are many other techniques for performing knee arthroplasty, this procedure often includes moving the knee cap to one side of the joint to expose the distal end of the femur and the proximal end of the tibia. FIG. 1 shows that in one non-limiting example of a TKR, the femur 100 and the tibia 105 are then cut and shaped to respectively receive a femoral prosthetic component 110 and a tibial prosthetic component 115. In this example, FIG. 1 shows the femur's distal end 120 can be cut to provide a flat distal surface 125, which is generally cut at a range from four to six degrees of valgus from the femur's anatomic axis (not illustrated). Additionally, FIG. 1 shows the femur's distal end 120 can also be cut to have a flat anterior surface 130 and a flat posterior surface 135. Moreover, FIG. 1 shows the femur's distal end 120 can also be cut to include a flat anterior chamfer 140 surface, which is disposed between the anterior surface 130 and the distal surface 125, and a flat posterior chamfer surface 145, which is disposed between the posterior surface 135 and the femur's distal surface 125. Additionally, FIG. 1 shows that in some TKR procedures, a flat cut is made across the proximal end 150 of the tibia 105 to provide a flat proximal-tibial surface 155. Depending on the design of the prosthesis that will be used, this cut is sometimes flat (perpendicular to the long axis) or is alternatively cut with a few degrees of posterior slope to match the knee's individual anatomy.

After the femur 100 and tibia 105 in the preceding example have been cut, FIG. 1 shows that trial prosthetic components 110 and 115 can be placed on the bones to ensure both that bones have been cut to the proper dimensions and that the knee can function properly with the trial components.

One of the primary goals in knee arthroplasty is to balance the gap between the cut surfaces of the femur and the tibia so that the gap between the surfaces, which is typically between about 7 and about 10 millimeters (“mm”), is the same when the joint is flexed (meaning the knee is bent) as when the joint is extended (meaning the leg is straightened). When these flexion/extension gaps are essentially the same size, the knee has essentially the same amount of play when the knee is flexed as it does when the knee is extended. Accordingly, balancing the flexion/extension gaps can help stabilize the knee and, thereby, provide a better chance of good clinical functioning, patient satisfaction, and component longevity.

Another primary goal in knee arthroplasty is to ensure that the knee is properly balanced from side to side so the knee does not have an excessive valgus (meaning a knock-kneed) or an excessive varus (meaning a bow-legged) configuration. Not only does balancing the knee's varus/valgus alignment provide the leg with a more aesthetically pleasing look, but this balancing also allows weight to be properly distributed across the articulating surfaces of the knee as it functions. As a result, balancing the knee's varus/valgus alignment can provide an even wear across prosthetic-articulating surfaces, increase component longevity, and improve the patient's sense of stability through range of motion.

Often, balancing the knee's flexion/extension gaps and/or varus/valgus alignment can be difficult. For instances, if a portion of one or more surfaces on the femur and/or the tibia are missing due to prior mechanical injury, bone deformation, inaccurate cutting, damage that occurred when a primary component was removed for a revision knee surgery, or some other factor, then the knee's flexion/extension gaps and/or the varus/valgus alignment may become imbalanced and prevent the knee from functioning properly.

When the knee's flexion/extension gaps and/or varus/valgus alignment are not balanced, there are several conventional methods that can be used to balance the gaps and/or straighten the knee's alignment. In one example, where the knee's flexion/extension gaps or varus/valgus alignment are unbalanced because too much bone is missing from a portion of the femur and/or the tibia, the high portion of the damaged bone is removed to balance the knee and one or more thicker prosthetic components are often used to compensate for the missing bone. In another example, where a missing portion of bone causes the knee's flexion/extension gaps and/or varus/valgus alignment to be unbalanced, a relatively large portion of the low end of the bone (or the portion missing desired bone) is removed with a step or slanted cut to allow a substantially square or large-wedged spacer to be placed between the bone and the prosthesis to properly balance the knee. These large augments are often attached to the prosthesis with screws, and typically begin at 4 mm heights. Furthermore, these large augments often fill an entire compartment (either medial or lateral) on either the tibial or femoral side of the knee. In still another example, the ligaments and other soft tissue around the knee are cut and otherwise loosened on the tight side of the knee to balance the gaps and/or to balance the knee's alignment. In yet another example, the low end of the bone is filled by adding cement between the low end of the bone and the prosthetic component that is attached to the bone.

While the aforementioned techniques may help balance the knee's flexion/extension gaps and/or varus/valgus alignment, such techniques are not necessarily without their shortcomings. Indeed, in one example, removing the high portion of the bone to balance the bone and using a larger prosthetic component to compensate for the missing bone can increase surgery time, expose the patient to additional trauma, present another opportunity for the bone to be cut inaccurately, increase operation costs, and result in more of the patient's native bone being removed, which may leave less bone for any possible surgeries or revision that may be needed in the future. In another example, similar shortcomings to those just mentioned may also be associated with removing more of the low end of the bone to allow a spacer (or large augment) to be inserted between the bone and the prosthetic component. In still another example, excessively loosening ligaments and static stabilizers around the knee may weaken and destabilize the knee. In yet another example, cement that is placed between the low end of the bone and the prosthetic component may squeeze out when pressure is applied to the knee. As a result, the proper spacing between the bone and the prosthetic component may not be maintained as the joint is assembled and pressure is applied to the knee. As a result of this last example, the final outcome of the component positioning may not be what the surgeon had anticipated when the surgeon checked the final alignment of the joint with the trial components before placing the actual prosthetic components in the joint.

Thus, while techniques currently exist that are used to balance the knee's flexion/extension gaps and varus/valgus alignment during knee arthroplasty, challenges still exist, including those mentioned above. Accordingly, it would be an improvement in the art to augment or even replace current techniques with other techniques.

Thus, some conventional configurations of spacers are only suitable to be placed between a single type of bone (e.g., a tibia) and a corresponding prosthetic joint component (e.g., a tibial tray). As well, some configurations of conventional spacers can only be placed in a single location between a bone and a prosthetic component seated on the bone.

In addition to the aforementioned benefits and advantages offered by certain embodiments of the shim 10, the shim may be beneficial for several other reasons. In one non-limiting example, unlike some known joint balancing methods that require additional bone to be cut to fit certain conventional spacers between a bone and prosthetic component seated thereon, the described shim can augment a bone and be used to balance a joint without requiring additional bone in the joint to be cut to fit the shim between the bone and the prosthetic component.

In another non-limiting example, some embodiments of the shim are sized and shaped so that they can be inserted into and removed from a crevice between a bone and a prosthetic component that is disposed at an end of the bone, when the component is seated on the bone. Thus, the shims can be used as trial shims, as previously discussed.

In still another non-limiting example, the shim retains the desired space between the bone and its corresponding prosthetic component until the cement hardens. Thus, the shim can prevent cement from being squeezed from a space between the bone and the prosthetic component when pressure is applied to the joint.

In still another non-limiting example, because the shim requires less ligament loosening and less bone removal than some conventional methods used for balancing a joint during arthroplasty, procedures using the described shim can reduce the probability of infection, can be performed faster, with less cutting, and be less traumatic than some conventional methods for balancing a joint.

In yet another non-limiting example, because the described shim is separate and independent from the prosthetic components it is intended to support, the described shim can be used with a wide variety of prosthetic components of different sizes, shapes, and manufacturers.

In still another non-limiting example, because the described shim does not need to be screwed in place, the shim can be relatively easy to use and require less bone modification than would be required if the shim needed a screw or another object to pass through the shim to secure the shim to the bone.

In yet another non-limiting example, because the shim can be placed at a variety of locations between a bone and a corresponding prosthetic component, the described shim can be used to raise a single corner of the prosthetic component to help balance the joint appropriately.

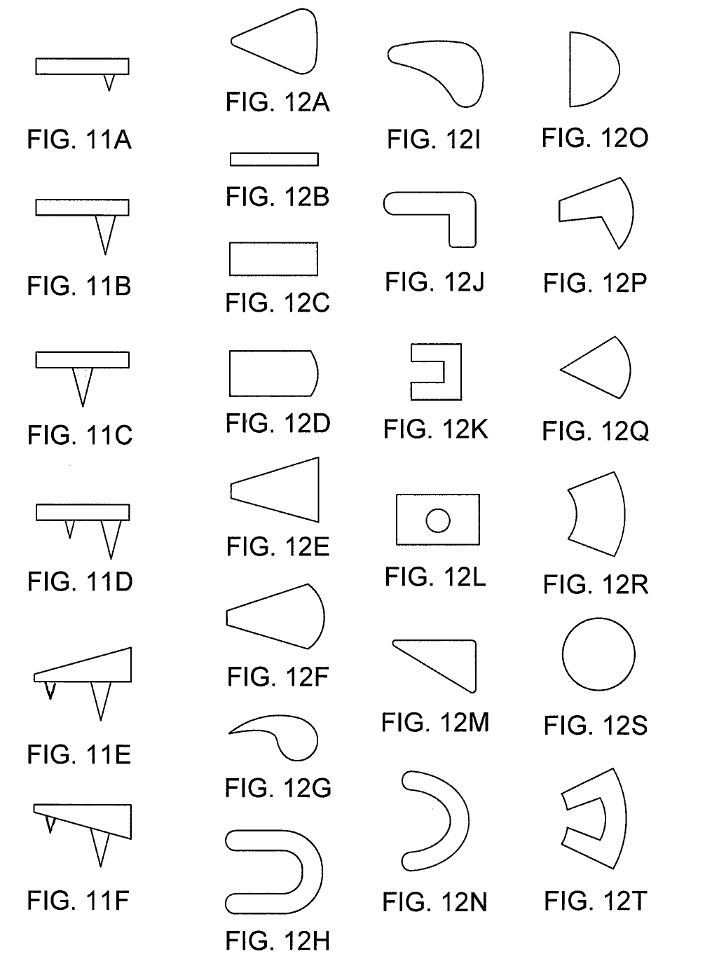

FIG. 11 illustrates a plurality of alternative exemplary means of arthroplasty shims. FIGS. 11A-F illustrates alternative exemplary embodiments of a spike or spikes extending from the surface of the shim to secure the shim in a desired location in relation to the bone. The number and position of the spike or spikes is selected based on the quality, quantity and characteristics of the bone being built up.

Alternative exemplary embodiments teach a shim comprising a plurality of spikes from zero spikes to several spikes. The spike-length may be uniform or varied. A spike may comprise a surface features such as a cavitated surface, or a plurality of small hooks or barbs, or one hook or barb, similar to a Velcro surface that allows the shim to be selectively placed or permanently placed on the bone.

According to certain alternative exemplary embodiments, the plurality of spikes may be positioned at any location of the surface of the shim, its edge, center or anywhere else. According to certain alternative exemplary embodiments, the shape of the spike may be conical, flat, tapered, untapered, conical from tip to the base or conical at the tip, curved, slanted, or narrow and needle-like in shape and appearance. The features selected, including, but not limited to the type, length, shape, style, feature or position of the spike will be determined by the type of bone congruent to the spike, the forces which will be applied to a spike or shim, the feature of other prosthesis in proximity to a spike.

Alternative exemplary embodiments teach a shim having a plurality of receivers on its surface wherein a spike may be selectively placed. Thus a shim may have a grid of receivers formed on its surface, and during a procedure or surgery the surgeon may identify a specific for securing the shim to a bone, the surgeon may then select the type of spike most effective for the type of bone and select the location on the shim which will be bring about the desired results. The surgeon may then selectively place the spike in the receiver to create the combination of spike features and position of the spike on the shim needed.

FIGS. 12A-T illustrate a plurality of alternative exemplary means for arthroplasty shims. In one exemplary embodiment a kit of shims comprising at least two of the shapes identified in FIG. 12A-T is provided either as trial or implant shims. A surgeon would select the appropriate shim from the kit based on factors such as the shape or space of the gap being filled, the forces that will be exerted on the joint, the patient's natural disposition, either varus, valgus, flexion or extension or any other factor that the surgeon considers relevant to the therapy.

The shim means disclosed in FIGS. 2-8 and 11 through 12 functions to build up the bone profile. A potential reason to build up the bone profile would be to achieve a desired shape or structural integrity. The exemplary shim embodiments can be sized appropriately for a bone restoration procedure in any sized patient, large or small.

The systems and methods for providing the shims for arthroplasty may be embodied in other specific forms without departing from its spirit or characteristics. All of the described embodiments and examples are to be considered in all respects only as illustrative and not as being restrictive. The scope of these systems and methods are, therefore, indicated by the appended claims rather than by the foregoing description.

1. A method of reducing the size of a gap formed between a bone and a prosthesis comprising:
identifying a gap between a bone and a prosthesis; and
placing at least one prosthetic shim into a gap formed between a bone and a prosthesis to reduce the size of the gap formed between the bone and the prosthesis.

2. The method of claim 1 wherein the shim is removed from the gap formed between the bone and the prosthesis without displacing the prosthesis.

3. The method of claim 1 wherein the shim is secured in place.

4. The method of claim 1 further comprising selecting a shim of appropriate shape and size as to fill a gap formed between a joint prosthesis and a bone.

5. The method of claim 1 further comprising building up a bone surface by securing the shim to the bone.

6. The method of claim 1 further comprising improving the structural integrity of the prosthesis by filling the gap with the shim.

7. An arthroplasty shim, comprising:
a biocompatible prosthetic shim means to build up a bone profile.

8. The arthroplasty shim of claim 7 wherein the shim is biocompatible.

9. The arthroplasty shim of claim 7 wherein the shim comprises materials which promote bone ingrowth.

10. The arthroplasty shim of claim 7 wherein the shim comprises surface chemistry which promotes bone ingrowth.

11. The arthroplasty shim of claim 7, wherein a perimeter of the shim defines a recess that is sized and shaped to fit around at least a portion of an object that extends from the prosthetic joint component into the bone.

12. The arthroplasty shim of claim 7, wherein the shim comprises a first face for contacting a surface of the bone and a second face for contacting the prosthetic joint component, and wherein a taper angle between the first face and the second face is between about 0.5 degrees and about 50 degrees.

13. The arthroplasty shim of claim 7, wherein the shim is capable of being temporarily placed in a crevice between the bone and the joint component when the prosthetic joint component is located at the joint end of the bone.

14. The arthroplasty shim of claim 7, further comprising a lip that extends from the peripheral end of the shim.

15. The arthroplasty shim of claim 7, further comprising at least one spike extending from a shim face.

16. The arthroplasty shim of claim 7, wherein the surface of a least a portion of the shim's surface comprises a cavitated surface.

17. The arthroplasty shim of claim 16 further comprising a chemically treated surface.

18. An arthroplasty shim, comprising:
a biocompatible shim further comprising at least one anchor feature that is sized, to retain the shim at a desired location on a bone;
wherein the shim is sized and shaped to fit between a first type of bone and a first prosthetic joint component at a plurality of locations when the first prosthetic joint component is located at a first joint end of the first type of bone;
wherein the shim comprises a maximum thickness between about 1 mm and about 5 mm; and
wherein the shim has a maximum width that is less than about 2 cm.

19. A method for balancing a joint by placing an arthroplasty shim between a bone and a prosthetic joint component, comprising:
providing an arthroplasty shim wherein the shim is sized to fit between the bone and the prosthetic joint component;
wherein the shim has a maximum thickness between about 0.5 mm and about 6 mm; and
selectively placing the shim on the bone.

20. The method of claim 19 further comprising driving a first amount of cement into the bone before securing the shim to the bone;

21. The method of claim 20 further comprising adding a second amount of cement between the bone and the prosthetic joint component.

22. The method of claim 19, further comprising placing the shim between the bone and the prosthetic joint component to balance a valgus/varus alignment of the joint.

23. The method of claim 19, further comprising placing the shim between the bone and the prosthetic joint component to balance a flexion gap with an extension gap in the joint.

24. The method of claim 19, further comprising strengthening the placement of the prosthetic joint component by placing the arthroplasty shim between the bone and the prosthetic joint component.

25. An applicator comprising:
a housing further comprising a shaft, one end of the shaft comprising a concave down surface so as to form a cup.
